(19) United States
(12) Patent Application Publication
(10) Pub. No.: US 2016/0129117 A1
(43) Pub. Date: May 12, 2016

(54) Novel Boronic Acid Compound Preparation

(71) Applicant: NIPPON KAYAKU KABUSHIKI KAISHA, Tokyo (JP)

(72) Inventors: Masatoshi Abe, Tokyo (JP); Masaharu Nakamura, Tokyo (JP); Osamu Miyazaki, Tokyo (JP); Keiko Sekine, Tokyo (JP)

(21) Appl. No.: 14/896,941

(22) PCT Filed: Jun. 27, 2014
(86) PCT No.: PCT/JP2014/067129

§ 371 Date: Dec. 9, 2015

(30) Foreign Application Priority Data
Jul. 3, 2013 (JP) 2013-139437

(51) Int. Cl. A61K 47/34; A61K 31/69

(52) U.S. Cl. CPC A61K 47/34; A61K 31/69

(57) ABSTRACT

The purpose of the present invention is to avoid side effects from contained medicines. Provided are: a preparation obtained by mixing a boronic acid compound and a block copolymer represented by general formula (I); and a production method therefor.

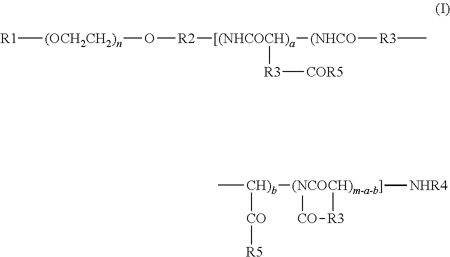

### TECHNICAL FIELD

The present invention relates to a novel preparation comprising a boronic acid compound and a block copolymer and use thereof.

### BACKGROUND ART

Peptide boronic acid compounds are widely known as serine/threonine protease inhibitors. Among these, the proteasome inhibitor, bortezomib (Trade Name: Velcade (Registered Trade Mark)) is clinically used as a therapeutic agent for multiple myeloma or mantle cell lymphoma. Other proteasome inhibitors such as delanzomib (CEP-18770) and ixazomib (MLN2238: the activity metabolite of MLN9708) are also known, and they have been developed as therapeutic agents for multiple myeloma. However, since proteasomes are also present in normal cells, side effects cannot be avoided. For example, there are known to be side effects of bortezomib such as peripheral nerve toxicity, gastrointestinal tract disturbances, bone marrow toxicity and so on. In particular, the peripheral nerve toxicity is clinically important.

For this reason, other methods of the administration of bortezomib than an intravenous injection of it have been studied in order to reduce its toxicity. Non-Patent Literature 1 reports the results of clinical test in subcutaneous administration of bortezomib. Non-Patent Literature 1 discloses that the subcutaneous administration of it inhibits increase of Cmax in blood, thereby reducing the peripheral nerve toxicity. In fact, the incidence of grade 3 peripheral neuropath; in the subcutaneous administration is 6%, which is less than 16% incidence in the intravenous administration. At present, the subcutaneous administration of bortezomib has been approved.

Non-Patent Literature 2 discloses that about 30 mg/m² of total dose of bortezomib causes the peripheral nerve toxicity. Therefore, if the pharmacokinetics of bortezomib is changed so as to accumulate a drug in bone marrow, thereby allowing the dose to be reduced, the peripheral nerve toxicity could be reduced.

As a method of changing pharmacokinetics, a method of preparing a high molecular DDS formulation is known. As high molecular DDS formulations of bortezomib, a liposome formulation and a micelle formulation are known.

Patent Literature 1 discloses that a liposomal formulation of bortezomib. This liposomal formulation has a polyol group in the inner core of liposome, to which polyol group a boronic acid group can be ester-linked. Patent Literature 1 discloses in vivo test examples using this liposomal formulation, but it fails to disclose the detailed experimental method and results of the tests. Therefore, pharmacokinetics, bioactivity such as antitumor activity or toxicity of the liposomal formulation are unclear.

Patent Literature 2 discloses a micelle having chemically-bound bortezomib. In Patent Literature 2, bortezomib is chemically bound to a carboxylic acid of a polyethylene glycol-polyglutamic acid-block copolymer via 4-(2,3-dihydroxy-3-phenylbutane-2-yl)benzylamine or 4-(2,3-dihydroxy-3-phenylbutane-2-yl)benzylamine.

Patent Literature 2 does not disclose accumulation of the chemically bound micelle in the bone marrow. As to its toxicity, Patent Literature 2 discloses that the micelle is expected to minimize Cmax, resulting in reduction of the toxicity, but the blood drug concentration, Cmax, of the chemically-bound micelle of the working example is higher. In addition, Patent Literature 2 discloses an in vivo antitumor test comparing an anti-tumor effect of the chemically-bound micelle on prostate cancer with that of bortezomib, but it fails to disclose the effect of said micelle on myeloma.

Patent Literatures 3 and 4 report a micelle preparation having a drug, such as doxorubicin hydrochloride, irinotecan hydrochloride, vincristine sulfate, docetaxel or indomethacin, physically-absorbed thereto. However, a micelle having bortezomib physically-absorbed thereto has not been reported. Accumulation of said micelle in the bone marrow has not been reported, either.

Patent Literature 5 relates to a micelle formulation having a proteasome inhibitor physically absorbed thereto. In Patent Literature 5, MG-12 is used as a proteasome inhibitor, and a polyethylene glycol-polyaspartic acid benzyl ester-block copolymer is used as the outer shell of the micelle. These are mixed in an organic solvent, and then the mixture is dialyzed with water to obtain the micelle formulation. However, Examples in Patent Literature 5 only describe MG-132 that is lipophilic. Although Patent Literature 5 mentions bortezomib as a proteasome inhibitor, it fails to disclose an example using bortezomib and an effect of the micelle on myeloma.

### CITATION LIST

### Patent Literature

Patent Literature 1: WO 2006/052733

Patent Literature 2: JP 5086497 B

Patent Literature 3: WO 2007/126110

Patent Literature 4: WO 2007/136134

Patent Literature 5: WO 2010/098265

Patent Literature 6: JP 4820758 B

Patent Literature 7: JP 5249016 B

Patent Literature 8: JP 3270592 B

### Non-Patent Literature

Non-Patent Literature 1: Lancet. Oncol. 2011; 12: 431-40.

Non-Patent Literature 2: Manual for handling disorders due to adverse drug reactions—Peripheral Nerve Disorders—, Ministry of Health, Labour and Welfare, Japan, 2009 May.

Non-Patent Literature 3: Bioorg. Med. Chem. Lett., p 33:3, Vol. 8 (1998)

### SUMMARY OF THE INVENTION

### Problems to be Solved by the Invention

The purpose of the preparation of bortezomib or analogs thereof of the present invention is to provide the effect equivalent to or more than bortezomib at lower dose and reduce side effects of it by accumulating it in the bone marrow.

### Means for Solving the Problems

The present invention is based on the fact that a novel preparation, which comprises a polyethylene glycol-polyamino acid block copolymer having a side chain of carboxy group esterified or amidated by a lipophilic functional group, and bortezomib, has a high ability of accumulation in the bone marrow and shows the efficacy equivalent to or more than bortezomib at lower dose.

Specifically, the present invention relates to the following (1)-(13).

(1) A preparation obtained by mixing a boronic acid compound with a block copolymer represented by the following general formula (I):

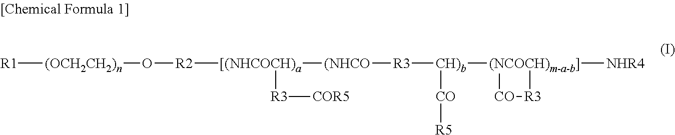

wherein

R1 represents hydrogen atom or a (C1-C5)alkyl group;

R2 represents a (C1-C5)alkylene group;

R3 represents methylene or ethylene group;

R4 represents hydrogen atom or a (C1-C4) acyl group;

R5 represents hydroxy group, an aryl(C1-C8)alkoxy group which may optionally have a substituent, or —(N6)—CO—NHR7;

R6 and R7, which may be the same or different from each other, represent a (C3-C6)cyclic alkyl group, or a (C1-C5) alkyl group which may be substituted by a tertiary amino group;

n represents 20-500;

m represents 2-200;

a represents 0-100;

b represents 0-100;

with proviso that the sum of a and b is 1 or more and no more than m, R5 is hydroxy group at a ratio of 0-5% relative to m, an optionally substituted aryl(C1-C8)alkoxy group at a ratio of 10-100% relative to m, and —N(R6)—CO—NHR7 at a ratio of 0-30% relative to m.

(2) The preparation wherein in the general formula (I), R1 is methyl group, R2 is n-propylene group, R3 is methylene group, R4 is acetyl group, n is 80-400, m is 15-60, a is 5-60, and b is 5-60.

(3) The preparation. wherein in the general formula (I), R1 is methyl group, R2 is n-propylene group, R3 is methylene group, R4 is acetyl group, n is 200-300, m is 30-60, a is 5-60, and b is 5-60.

(4) The preparation wherein in the general formula (1), both R6 and R7 are cyclohexyl, ethyl or isopropyl group, or either one of R6 or R7 is ethyl group and the other one is dimethylaminopropyl group.

(5) The preparation wherein in the general formula (1), each of R6 and R7 is isopropyl group.

(6) The preparation wherein the boronic acid compound is bortezomib, or an analog or a pharmacologically acceptable salt thereof.

(7) The preparation obtained by mixing a solution of bortezomib or an analog thereof with a solution of the block copolymer.

(8) The preparation wherein the solvent of the solution of bortezomib or the analog thereof and the solution of the block copolymer is ethanol.

(9) A method for producing the preparation comprising the following steps:

(a) dissolving a boronic acid compound and a block copolymer in a solvent;

(b) stirring the solution of the boronic acid compound and the block copolymer under heating; and

(c) stirring the solution of the boronic acid compound and the block copolymer while slow cooling;

(10) A medicament comprising the preparation.

(11) A therapeutic agent for a malignant disease, comprising the preparation.

(12) A therapeutic agent for a boric marrow-associated disease comprising the preparation.

(13) A therapeutic agent for multiple myeloma comprising the preparation.

### Effects of the Invention

The preparation of the invention is a preparation obtained by mixing a boronic acid compound with a block copolymer of polyethylene glycol and polyglutamic acid or polyaspartic acid, said block copolymer having an allylalcohol group ester bound to a side chain carboxy group or an urea derivative bound to said carboxy group. The preparation of the invention forms nanoparticles, allowing the drug to accumulate in the bone marrow. Therefore, the preparation of invention is expected to enhance the efficacy of the drug and reduce the toxicity of the drug (in particular, the peripheral nerve toxicity) due to reduction of the dose of the preparation.

### BRIEF DESCRIPTION OF THE DRAWINGS

FIG. 1 illustrates the results of measuring the amount of M protein in plasma of mice of each group of administration on day 23 in a test for antitumor activity of the compounds of Examples on multiple myeloma (Test example 2).

### MODE FOR CARRYING OUT THE INVENTION

The preparation of the invention is obtained by mixing a boronic acid compound with a block copolymer represented by said general formula (I), wherein R1 represents hydrogen atom or a (C1-C5)alkyl group;

R2 represents a (C1-C5)alkylene group;
R3 represents methylene or ethylene group;
R4 represents hydrogen atom or a (C1-C4) acyl group;
R5 represents hydroxy group, an optionally substituted aryl(C1-C8)alkoxy group, or —N(R6)—CO—NHR7;
R6 and R7, which may be the same or different from each other, represent a (C3-C6) cyclic alkyl group, or a (C1-C5) alkyl group which may be substituted by a tertiary amino group;
n represents 20-500;
m represents 2-200;
a represents 0-100;
b represents 0-100;
with proviso that the sum of a and b is 1 or more and no more than m, and R5 is a hydroxy group at a ratio of 0-5% relative to m, an optionally substituted aryl(C1-C8)alkoxy group at a ratio of 10-100% relative to m, and —N(R6)—CO—NHR7 at a ratio of 0-30% relative to m.

In the general formula (I) according to the invention, R1 is hydrogen atom or a (C1-C5)alkyl group. Examples of the (C1-C5)alkyl group include methyl, ethyl, n-propyl, isopropyl, n-butyl, s-butyl, t-butyl, n-pentyl groups and the like. R1 is preferably methyl group.

Examples of the (C1-C5)alkylene group of R2 include methylene, ethylene, n-propylene, n-butylene groups and the like. The (C1-C5) alkylene group is preferably ethylene or n-propylene group.

R³ is methylene or ethylene group, preferably methylene group.

R4 is hydrogen atom or a (C1-C4)acyl group, preferably a (C1-C4)acyl group. Examples of the (C1-C4)acyl group include formyl, acetyl, propionyl, butyroyl groups and the like. Among these, acetyl group is in particular preferable.

In the general formula (I), the aryl(C1-C8)alkoxy group of R5 is a linear or branched (C1-C5)alkoxy group to which an aromatic hydrocarbon group, such as phenyl or naphthyl group, is attached to. Examples of the aryl(C1-C8)alkoxy group include benzyloxy, phenethyloxy, phenylpropoxy, phenylbutoxy, phenylpentyloxy, phenylhexyloxy, phenylheptyloxy, phenyloctyloxy, naphthylethoxy, naphthylpropoxy, naphthylbutoxy, naphthylpentyloxy groups and the like.

Examples of the substituents on the aryl(C1-C8)alkyloxy group which may optionally have a substituent include lower alkoxy groups such as methoxy, ethoxy, isopropoxy, n-butoxy and t-butoxy, halogen atom such as fluorine, chlorine and bromine, nitro group, cyano group and the like. The number of substituents may be 1 to the maximum number of substituents substituted at all possible positions. Preferably, the aryl(C1-C8)alkoxy group is not substituted.

The aryl(C1-C8)alkoxy group which may optionally have a substituent is preferably an unsubstituted phenyl (C1-C6)alkoxy group. Examples thereof include an unsubstituted benzyloxy group, an unsubstituted phenethyloxy group, an unsubstituted phenylpropoxy group, an unsubstituted phenylbutoxy group, an unsubstituted phenylpentyloxy group, an unsubstituted phenylhexyloxy group and the like. Among these, an unsubstituted benzyloxy group and an unsubstituted phenylbutoxy are in particular preferable.

One of the substituents represented by R5 is —N(R6)—CO—NHR7. The substituents R6 and R7 represent a (C3-C6)cyclic alkyl group or a (C1-C5)alkyl group which may be substituted by a tertiary amino group. Examples of the (C3-C6)cyclic alkyl group and the (C1-C5)alkyl group which may be substituted by a tertiary amino group include cyclopropyl, cyclopentyl, cyclohexyl, methyl, ethyl, isopropyl, n-butyl, 3-dimethylaminopropyl and 5-dimethylaminopentyl groups, and the like. Among these, ethyl, isopropyl, cyclohexyl or 3-dimethylaminopropyl group is preferable, and isopropyl group is particularly preferable.

In the general formula (1), n represents 20-500, preferably 80-400, particularly preferably 200-300. m represents 2-200, preferably 15-60, particularly preferably 30-60. Each of a and b represents 0-100, preferably 5-60. The sum of a and b is 1 or more and no more than m.

In the general formula (1), m means the polymerization number of the amino acid structural unit in the polyamino acid structural moiety. The polyamino acid structural moiety comprises each structural unit wherein R5 in the general formula (I) is hydroxy group, an optionally substituted aryl(C1-C8)alkoxy group, or —N(R6)—CO—NHR7, and the structural unit forming a cyclic imide structure.

R5 in the general formula (I) is hydroxy group at the ratio of 0-5%, preferably 0-3% relative to m, an optionally substituted aryl(C1-C8)alkoxy group at the ratio of 10-100%, preferably 20-80% relative to m, and —N(R6)—CO—NHR7 at a ratio of 0-30% relative to m.

In the block copolymer represented by the general formula (I), the ratio at which R5 is hydroxy group is particularly preferably 0% relative to m, 0% of the ratio at which R5 is hydroxy group relative to m means that all the carboxy groups of the polyamino acid structural moieties in the compound of the general formula (I) are substituted by the optionally substituted aryl(C1-C8)alkoxy group and/or —N(R6)—CO—NHR7.

In the polyamino acid structural moiety of the block copolymer represented by the general formula (I) used in the invention, each amino acid structural unit may be bound randomly or may be bound to form a block type. Therefore, the polyamino acid structure expressed in the general formula (I) is merely one example. For example, the block copolymers respectively represented by the following general formulae (II)-1 and -2 are also included in the block copolymers used in the invention.

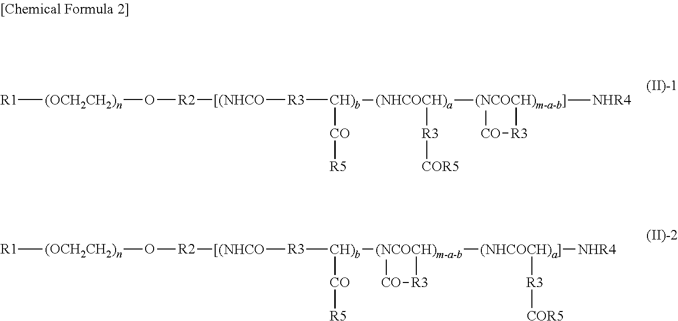

An optionally substituted aryl(C1-C8)alkylalcohol in the invention an alcohol corresponding to said optionally substituted aryl(C1-C8)alkoxy group.

As the optionally substituted aryl(C1-C8)alkylalcohol, the corresponding commercially-available compounds, the corresponding compounds prepared by the known organic synthesis methods, or the corresponding compounds prepared by applying the known organic reactions may be used.

The boronic acid compound in the invention is not limited as long as it is a compound having boronic acid group or a boronic acid ester group, or a trimeric compound formed by the dehydration of a boronic acid group. Preferably, the boronic acid compound has an activity of inhibiting a proteasome.

Bortezomib or analogs thereof include bortezomib, bortezomib trimer, or bortezomib ester represented by the following general formula (III):

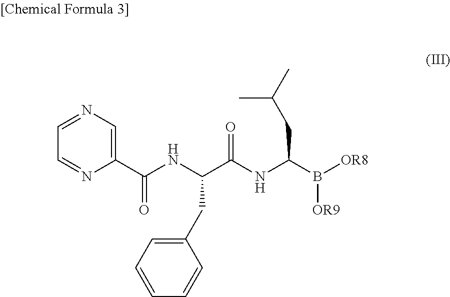

wherein
R8 and R9, which may be the same or different from each other, are respectively an optionally substituted. (C1-C5)alkyl group or an optionally substituted (C3-C7)cyclic alkyl group; or R8 and R9 may be linked together to form an optionally substituted (C1-C5)alkyl group or an optionally substituted (C3-C7)cyclic alkyl group. Examples of the bortezomib ester include a dimethyl ester, diethyl ester, a di(n-propyl)ester, a diisopropyl ester, a cyclonexanediol ester, a pinanediol ester and the like. Among these, a diethyl ester and a pinanediol ester are particularly preferable.

The present invention includes a process for producing the preparation of the invention. The preparation of the invention can be obtained by stirring bortezomib or an analog thereof and the block copolymer represented by the general formula (I) in a solvent. A solvent to be used is not particularly limited. as long as it is the solvent in which bortezomib or an analog thereof and the block copolymer represented by the general formula (I) are together soluble and which can be distilled off under reduced pressure. Examples or the solvent include alcohols such as methanol, ethanol and propanol, and acetonitrile etc. The solvent is preferably ethanol. Moreover, the amount of the drug contained in the preparation of the invention is 1 to 50 mass % per a total preparation, preferably 3 to 15 mass %. The reaction temperature in the stirring ranges from 30 to 50° C. The time for stirring ranges from 0.1 to 10 hours, Preferably, in the stirring, the block copolymer and the drug are mixed at 35 to 45° C., followed by slow cooling of a mixture to 10 to 25° C. After the slow cooling, the solvent is removed by conventional methods to give the preparation of the invention.

The preparation of the invention can be used as a pharmaceutical agent which is indicated, for a disease for which the physiologically active substance contained in the preparation preparation of the invention may be used in a dosage form which is conventionally used, including injections, tablets, and powders. The preparation of the invention may contain conventionally-used pharmaceutically acceptable carriers including, for example, binding agents, lubricating agents, disintegrating agents, solvents, vehicles, solubilizing agents, dispersing agents, stabilizing agents, suspending agents, preservatives, soothing agents, colorants, flavors and the like. The preparation of the invention may be used via a conventionally-used process using these ingredients.

The preparation of the invention is preferably used as an injection, and usually water, a physiological saline, a 5% glucose or mannitol liquid, a water-soluble organic solvent (for example, glycerol, ethanol, dimethyl sulfoxide, N-methylpyrrolidone, polyethylene glycol, cremophor, and a mixture thereof) or a mixture of water and the water-soluble organic solvents may be used.

The dosage of the preparation of the invention may vary as a matter of course, depending on the characteristics of physiologically active substance as well as the sex, age, physiological condition, pathological condition and the like of a patient. The preparation is parenterally administered, typically at a dose of 0.01 to 500 mg/m², preferably 0.1 to 100 mg/m², particularly preferably 0.1 to 10 mg/m² as an active ingredient per day for an adult. For example, the administration by injection may be performed intravenously, intra-arterially, subcutaneously, or into an affected site (a tumor site).

### EXAMPLE

Hereinafter, the invention will be illustrated more specifically with reference to Examples. However, the scope of the invention is not limited to these Examples. The Gaussian distribution analysis for measuring the size of the particles (i.e. particle diameter) that are constituted by the product of the invention in an aqueous solution was conducted by using a ZetaPotential/Particlesizer NICOMP (Registered Trademark) 380ZLS, manufactured by Particle Sizing Systems Co. (Equipment A) or particle size and zeta potential measurement device, Zetasizer Nano ZS manufactured by Malvern Instruments Ltd (Equipment B).

### Production of Polymer A

Polymer A was synthesized according to Reference Example 1 in Patent Literature 7.

A methoxy polyethylene glycol having an aminopropyl group at the end (SUNBRIGHT MEPA-12T, manufactured by Nippon Oil & Fats Co., Ltd., average molecular weight 12,000, 1.0 g) was dissolved in DMSO (20 mL), and β-benzyl L-aspartate N-carboxylic anhydride (0.94 g) was then added thereto. The mixture was stirred for 20 hours at 35° C. Ethanol (40 ml) and diisopropylether (160 mL) were added to the reaction liquid, and the mixture was stirred for 90 minutes at room temperature. Then, the precipitate was collected by filtration and washed with ethanol/diisopropylether (1/4 (v/v 50 mL).

The resultant precipitate was dissolved in DMF (20 mL), and acetic anhydride (0.3 mL) was added. The mixture was stirred for 15 hours at room temperature. Ethanol (40 mL) and diisopropylether (160 mL) were added to the reaction liquid, and the mixture was stirred for 90 minutes at room temperature. Then, the precipitate was collected by filtration, and washed with ethanol/diisopropylether (1/4(v/v), 50 mL) to obtain a solid of Polymer A.

### Production of Polymer B

Polymer B was synthesized according to Example 1 of Patent Literature 6.

N-acetylated poly(ethylene glycol)-poly(aspartic acid) block copolymer (PEG (average molecular weight 12,000)-PAsp(polyaspartic acid; average polymerization degree 40)-Ac) (represented the following general formula (IIV) wherein R1 is a methyl group. R2 is trimethylene group, R3 is a methylene group, R4 is an acetyl group, n is about 272, a is about 10, b is about 30, abbreviated hereinafter as PEG-pAs-Ac) was obtained according to Example 1 of Patent Literature 8.

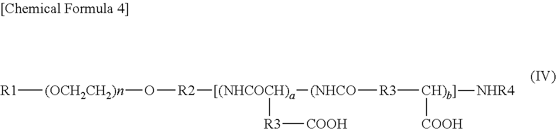

The definitions of R1, R2, R3, R4, n, a and b an the general formula (IV) are identical to those in the general formula (I).

Next, DMAP, 4-phenyl-1-butanol and DIPCI were added to the obtained PEG-pAsp-Ac and reacted to obtain a block copolymer. Further, DMAP and DIPCI were added to the obtained block copolymer and reacted, and then purified by use of a cation-exchange resin, Dowex 50w8, to obtain Polymer B.

### Analysis of Polymer B

Polymer B (27.6 mg) was dissolved in 2 ml of acetonitrile, and 2 ml of 0.5N aqueous sodium hydroxide solution was added thereto. The solution Was stirred for 20 minutes at room temperature to hydrolyze its ester linkage, then neutralized with 0.5 mL of acetic acid, and prepared to a volume of 25 ml with 50% hydrous acetonitrile. The prepared solution was quantified for free 4-phenyl-1-butanol by reverse HPLC. The result indicated that 4-phenyl-1-butanol bound via an ester linkage was 49% relative to m (the polymerization number of the polyaspartic acid structural moieties of the block copolymer) in the general formula (I).

Then, measuring Polymer B by anion exchange HPLC under conditions as described below, no peak retained on the column was detected.

### Measurement Conditions for Anion Exchange HPLC

Column: TSKgel DEAE-5PW (manufactured by Tosoh Corporation)
Sample concentration: 10 mg/mL
Injection volume: 20 μL
Column temperature: 40° C.

### Mobile Phases

(A) 20 mM Iris-hydrochloric acid buffer (pH 8.0): acetonitrile=80:20
(B) 20 mM Iris-hydrochloric acid buffer+1M aqueous sodium chlorides solution (pH 8.0): acetonitrile=80:20
Flow rate: 1 mL/min
Gradient condition B % (min): 10 (0), 10 (5), 100 (40), 10 (40.1), stop (50.1)
Detector: UV-visible spectrophotometric detector (detection wavelength 260 nm)

Polymer B was dissolved in a mixed solution of deuterated sodium. hydroxide (NaOD)-heavy water (D₂O)-deuterated acetonitrile (CD₃CN), and measured by NMR, indicating that the partial structure of —N(i-Pr)-CO—NH(i-Pr) (corresponding to a structure of the N(R6)—CO—NHR7 in the general formula (1) wherein each of R6 and R7 is an isopropyl group) was 14% relative to m.

### Reference Example 1

### Synthesis of Bortezomib

n-Hexane (150 mL), acetonitrile (207 mL), 1N hydrochloric acid (23 ml) and phenyl boronic acid (1.01 g) were added to bortezomib (1S,2S,3R,5S)-pinanediol ester (3.58 g) synthesized according to the method disclosed in Non-Patent Literature 3. Then, the mixture was stirred for 1.5 hours at room temperature, and an upper layer of n-hexane was removed. n-Hexane (150 mL) was added to this solution, then the mixture was stirred for 1 hour, and an upper layer of n-hexane was removed. This operation was repeated three times (addition of n-hexane (150 mL) and then stirring for 1 hour, addition of n-hexane (150 mL) and then stirring of 15.5 hours, and addition of n-hexane (100 mL) and then stirring for 0.75 hours).

The solution of n-hexane in the reaction solution was removed, and then the solvent was distilled off under reduced pressure. 50% aqueous acetone solution (30 mL) and acetonitrile (6 mL) were added to the residue. Then, the residue was dissolved in the media, and it purified with DIAION HP20 column chromatography (450 mL, gradient elution with water-50% aqueous acetone solution). Acetone in the eluate was distilled off under reduced pressure until just before crystal precipitation, and it was freeze dried to obtain the compound (1.90 g) mentioned in the title.

### Reference Example 2

### Synthesis of Bortezomib Trimer

Acetonitrile (3 ml) was added to bortezomib (300 mg) obtained, in Reference Example 1, and the mixture was stirred at room temperature. Once bortezomib was dissolved, precipitation of crystal was confirmed. Then, diisopropylether (3 ml) was dropped slowly, and the mixture was stirred for 10 minutes at room temperature. The crystal was collected by filtration, and was dried under reduced pressure to obtain the compound (254 mg) mentioned, in the title.

### EXAMPLE 1

### Bortezomib Preparation (30/B570)

Ethanol (2.85 mL) was added to the bortezomib trimer (30 mg) and Polymer B (570 mg), and the mixture was stirred for 6 hours at the external temperature of 40° C. Then, the mixture was gradually cooled to the external temperature of 20° C. with stirring, thereby causing solidification of the mixture. The solvent of ethanol was distilled off at room temperature under reduced pressure to obtain a bortezomib preparation (30/B570). The content of bortezomib: 4.1%. Particle diameter (Equipment B): 56 nm (Z-Average).

### EXAMPLE 2

### Bortezomib Preparation (30/B300)

Ethanol (1.50 mL) was added to the bortezomib trimer (30 mg) and Polymer B (300 mg), and the mixture was stirred for 6 hours at the external temperature of 40° C. Then, the mixture was gradually cooled to the external temperature of 20° C. with stirring, thereby causing solidification of the mixture. The solvent of ethanol was distilled off at room temperature under reduced. pressure to obtain a bortezomib preparation (30/B300). The content of bortezomib: 7.0%. Particle diameter (Equipment B): 58 nm (Z-Average).

### EXAMPLE 3

### Bortezomib Preparation (30/B170)

Ethanol (0.85 ml) was added to the bortezomib trimer (30 mg) and Polymer B (170 mg), and the mixture was stirred for 6 hours at the external temperature of 40° C. Then, the mixture was gradually cooled to the external temperature of 20° C. with stirring, thereby causing solidification of the mixture. The solvent of ethanol was distilled off at room temperature under reduced pressure to obtain a bortezomib preparation (30/B170). The content of bortezomib: 12%. Particle diameter (Equipment B): 54 nm (Z-Average).

### EXAMPLE 4

### Bortezomib (1S,2S,3R,5S)-pinanediol Ester Formulation (20/B200)

Ethanol (1 ml) was added to bortezomib (1S,2S,3R,5S)-pinanediol ester (20 mg) and Polymer B (200 mg), and the mixture was stirred for 2 hours at the external temperature of 40° C. Then, the mixture was gradually cooled and stirred at room temperature, thereby causing solidification of the mixture. The solvent, of ethanol was distilled off at room temperature under reduced pressure to obtain a preparation of bortezomib (1S,2S,3R,5S)-pinanediol ester (20/200). The content of bortezomib: 5.1%. Particle diameter (Equipment A): 40 nm (Volume).

### EXAMPLE 5

### Bortezomib Preparation (20/A200)

Ethanol (1 ml) was added to the bortezomib trimer (20 mg) and Polymer A (200 mg), and the mixture was stirred for 4 hours at the external temperature of 40° C. Then, the mixture was gradually cooled to the external temperature of 20° C. with stirring, thereby causing solidification. The solvent of ethanol was distilled off at room temperature under reduced pressure to obtain a bortezomib preparation (20/A200). The content of bortezomib: 8.1%. Particle diameter (Equipment B): 58 nm (Z-Average).

### Comparative Example 1

### Preparation of Bortezomib and (D)-Mannitol

The bortezomib trimer (20 mg) and (D)-mannitol (200 mg) were dissolved in acetonitrile (2 mL), and then water (10 mL) was added to the mixture. The mixture was freeze dried to obtain the preparation mentioned in the title. The content of bortezomib 9.47%.

### Comparative Example 2

### Micelle Including Bortezomib According to Patent Literature 5 (5/B20)

A micelle including bortezomib according to Patent Literature 5 was produced according to the steps and the conditions described in Example 1 of Patent Literature 5. The solution of the bortezomib trimer (5.2 mg) in dimethylsulfoxide (10 mL), and the solution of Polymer B (20.2 mg) in dimethylsulfoxide (10 mL) were mixed. The mixture was stirred for minutes at room temperature (internal temperature: 25° C.) Then, water (5 ml) was added to this solution, and the mixture was stirred for 10 minutes at room temperature (internal temperature was elevated to 34° C., and the solution became clouded to precipitate a solid). In addition, water (5 mL) was added to the mixture, and the mixture was stirred for 10 minutes room temperature (the precipitated solid was not dissolved). Adding water (5 mL) to the mixture and stirring the mixture for 10 minutes at room temperature was repeated twice. Then, water (10 mL) was added to the mixture at room temperature and stirred for 20 minutes (at this time, the precipitated solid was dissolved). Water (10 mL; total 40 mL) was further added to the mixture, and the mixture stirred for 20 minutes at room temperature. It was confirmed in Equipment A that the reaction solution formed particles, and a peak of bortezomib was confirmed in HPLC (the amount of injection: 6 μL). 20 mL of water (total 60 mL) was added to the solution, after which the solution was dialyzed from water (2 L) with a dialysis membrane (MW: 1000). The dialysis was conducted (at room temperature for 24 hours; the external solution of water was exchanged six times) until a peak of dimethylsulfoxide was almost disappeared in HPLC. After the completion of the dialysis, it was confirmed in HPLC for the internal solution of the dialysis membrane and its wash solution (150 mL) (the amount of injection: 15 μL) that a peak of bortezomib disappeared.

### Comparative Example 3

### Micelle Including Bortezomib According to Patent Literature 5 (5/A20)

A micelle including bortezomib according to Patent Literature 5 was produced according to the steps and the conditions described in Example 1 of Patent Literature 5. The solution of the bortezomib trimer (5.0 mg) in dimethylsulfoxide (1.0 mL), and the solution of Polymer A (19.9 mg) in dimethylsulfoxide (10 mL) were mixed. The mixture was stirred for 10 minutes at room temperature. Then, water (10 mL) was each added forth times to the mixture, after which it was confirmed in Equipment A that the reaction solution formed particles like Comparative example 2. 20 mL of water (total 60 mL) was added to the solution, after which the solution was dialyzed from water (2 L) with a dialysis membrane (MW: 1000). The dialysis was conducted (at room temperature for 24 hours; the external solution was exchanged six times) until a peak of dimethylsulfoxide almost disappeared in HPLC. After the completion of dialysis, the internal solution of the dialysis membrane and its wash solution (150 mL) were confirmed in HPLC (the amount of injection: 15 μL), and a peak of bortezomib was found to be disappeared.

### Test Example 1

1 mL of aqueous solution of each preparation of Examples 2 and 5 was prepared to have a concentration of 1 mg/mL expressed in terms of polymer. Each of the aqueous solution was dialyzed from 1 L of water with the dialysis membrane (MW: 1000). Each amount of bortezomib on the inside of the dialysis membrane before, after 3 hours and after 27 hours of the dialysis was analyzed in HPLC. The results are shown in Table 1.

TABLE 1
HPLC area of the amount of bortezomib on the inside of the
dialysis membrane

| | | Before the | After 3 hours | After 27 hours |
|---|---|---|---|---|
| | | dialysis | of the dialysis | of the dialysis |
| Example 2 | HPLC area | 1349196 | 565042 | 0 |
| | Percentage | 100% | 41.9% | non- |
| | to the | | | detectable: 0% |
| | preparation | | | |
| | before | | | |
| | the dialysis | | | |
| Example 5 | HPLC area | 1079497 | 507277 | 0 |
| | Percentage | 100% | 47.0% | non- |
| | to the | | | detectable: 0% |
| | preparation | | | |
| | before | | | |
| | the dialysis | | | |

After 3 hours of the dialysis, 50% or more of bortezomib in both preparations was dispersed into the external solution. After 27 hours of the dialysis, all bortezomib in both preparations was dispersed into the external solution.

From these results, it was confirmed that the bortezomib preparations of the invention appropriately released bortezomib. Meanwhile, in the formulating method disclosed in Patent Literature 5 using the dialysis, the dialysis removing dimethylsulfoxide allows bortezomib to effuse out of the micelle. Therefore, this indicates that the conditions under which dimethylsulfoxide is removed cannot isolate the micelle containing bortezomib. In order to obtain the preparation comprising bortezomib and the block copolymer, the formulating method of the invention is more appropriate wherein bortezomib and the block copolymer are dissolved in the solvent such as ethanol which allowing them to be dissolved under heating, then they are cooled, and the solvent is removed under reduced pressure.

### Test Example 2

### Test for Antitumor Activity of the Compounds of Examples (Multiple Myeloma)

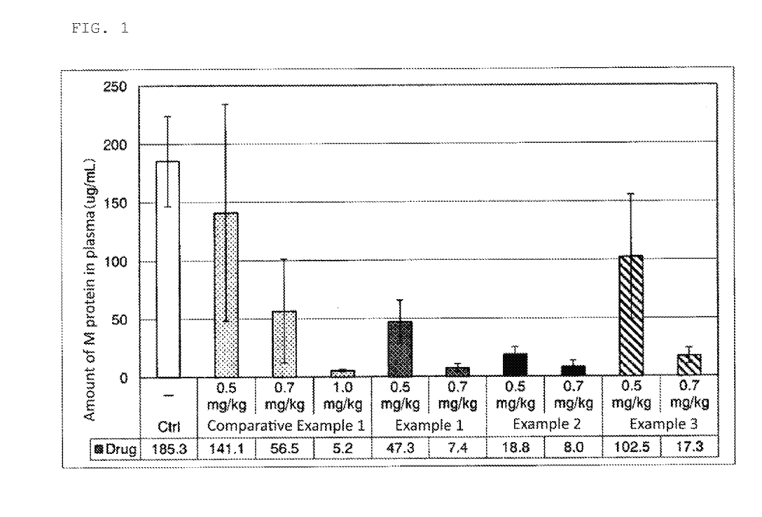

Human multiple myeloma MM.1S (Cell number: 3×10⁶) was intravenously injected into tail veins of SCID mice (Japan Charles River Laboratories Japan: 6 weeks of age). An amount of M protein in plasma was measured after 4 weeks, and the mice were grouped into a group of mice having an average value of 0.96 μg/mL (3 to 4 mice per group). Then, the preparations of Examples 1-3 and Comparative example 1 (bortezomib preparation) were dissolved in 5% glucose solution, and they were administered from the tail vein at day 0, 3, 7 and 10. Furthermore, as a group of negative control, 5% glucose solution was administered according to the same schedule. The doses of the preparation of Comparative example 1 were 1, 0.7 and 0.5 mg/kg, and the doses of the preparations of Example 1-3 were 0.7 and 0.5 mg/kg. The amount of M protein in plasma at day 23 of each group of mice was measured. The results are shown in FIG. 1.

As the results of the test for antitumor activity, it was confirmed that the amount of M protein in plasma (IgE antibody level) of the group of negative control (Control) was 185 μg/mL, and the amount of M protein in plasma was increased with the growth of myeloma cells MM.1S. In contrast, The amount of M protein in the group of administration of 1 mg/kg of bortezomib (D)-mannitol preparation of Comparative example 1 is 5.2 μg/mL, the amount in the group of administration of 0.7 mg/kg is 57 μg/mL, and the amount of administration of 0.5 mg/kg is 141 μg/mL, indicating a dose-dependent anti-tumor effect. Meanwhile, for the bortezomib preparation of Example 2, the amount of the protein in the group of administration of 0.7 mg/kg is 8.0 μg/mL, and the amount in the group of administration of 0.5 mg/kg is 19 μg/mL, indicating a dose-dependent and strong anti-tumor effect. The effect is stronger than that of the preparation of Comparative example 1. Furthermore, the bortezomib preparations of Examples 1 and 3 also showed a stronger anti-tumor effect than that of the preparation of Comparative example 1.

From the above results of antitumor test, it was confirmed that the preparation of the invention accumulates in bone marrow more and showed a stronger anti-tumor effect than those of the bortezomib (D)-mannitol preparation.

1. A preparation obtained by mixing a boronic acid compound with a block copolymer represented by the following general formula (I): 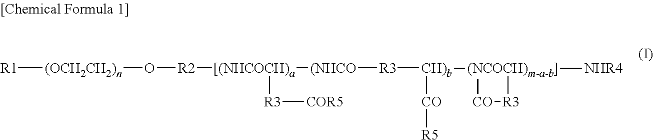
wherein
R1 represents hydrogen atom or (C1-C5)alkyl group,
R2 represents (C1-C5)alkylene group,
R3 represents methylene or ethylene group;
R4 represents hydrogen atom or (C1-C4) acyl group;
R5 represents hydroxy group, aryl(C1-C8)alkoxy group which may optionally have a substituent, or —N(R6)—CO—NHR7;
R6 and R7, which may be the same or different from each other, represent (C3-C6)cyclic alkyl group, or (C1-C5)alkyl group which may be substituted by a tertiary amino group;
n represents 20-500;
m represents 2-200;
a represents 0-100;
b represents 0-100;
with proviso that the sum of a and b is 1 or more and no more than m, R5 is a hydroxy group at a ratio of 0-5% relative to m, optionally substituted aryl(C1-C8)alkoxy group at a ratio of 10-100% relative to m, and —N(R6)—CO—NHR7 at a ratio of 0-30% relative to m.

2. The preparation according to claim 1, wherein in the general formula (I), R1 is methyl group, R2 is n-propylene group, R3 is methylene group, R4 is acetyl group, n is 80-400, m is 15-60, a is 5-60, and b is 5-60.

3. The preparation according to claim 1, wherein in the general formula (I), R1 is methyl group, R2 is n-propylene group, R3 is methylene group, R4 is acetyl group, n is 200-300, m is 30-60, a is 5-60, and b is 5-60.

4. The preparation according to claim 1, wherein in the general formula (I), both R6 and R7 are cyclohexyl, ethyl or isopropyl group, or either one of R6 or R7 is ethyl group and the other one is dimethylaminopropyl group.

5. The preparation according to claim 4, wherein in the general formula (I), each of R6 and R7 is isopropyl group.

6. The preparation according to claim 1, wherein the boronic acid compound is bortezomib or an analog thereof.

7. The preparation according to claim 1 obtained by mixing a solution of bortezomib or analogs thereof with a solution of the block copolymer.

8. The preparation according to claim 7, wherein the solvent of the solution of bortezomib or the analog thereof and the solution of the block copolymer is ethanol.

9. A method for producing the preparation according to claim 1, comprising the following steps:
(a) dissolving bortezomib or an analog thereof and a block copolymer in a solvent;
(b) stirring the solution of bortezomib or the analog thereof and the block copolymer under heating; and
(c) stirring the solution of bortezomib or the analog thereof and the block copolymer while slow cooling.

10. A medicament comprising the preparation according to claim 1.

11. A therapeutic agent for a malignant disease, comprising the preparation according to claim 1.

12. A therapeutic agent for a bone marrow-associated disease comprising the preparation according to claim 1.

13. A therapeutic agent for multiple myeloma comprising the preparation according to claim 1.

14. The preparation according to claim 2, wherein the boronic acid compound is bortezomib or an analog thereof.

15. The preparation according to claim 3, wherein the boronic acid compound is bortezomib or an analog thereof.

16. The preparation according to claim 4, wherein the boronic acid compound is bortezomib or an analog thereof.

17. The preparation according to claim 5, wherein the boronic acid compound is bortezomib or an analog thereof.
